(19) United States
(12) Patent Application Publication
(10) Pub. No.: US 2025/0154189 A1
(43) Pub. Date: May 15, 2025

(54) Modified Sophorolipids with Enhanced Defoaming Properties

(71) Applicant: Locus Solutions IPCo, LLC, Solon, OH (US)

(72) Inventors: Lee SPEIGHT, Solon, OH (US); Andrew MORRIS, Richmond, VA (US); Daniel HAGAMAN, Mechanicsville, VA (US)

(21) Appl. No.: 18/839,504

(22) PCT Filed: Jul. 12, 2023
(86) PCT No.: PCT/US2023/070033

§ 371 Date: Aug. 19, 2024

Related U.S. Application Data
(60) Provisional application No. 63/388,419, filed on Jul. 12, 2022.

(51) Int. Cl. C07H 15/10; C07H 9/02; C07H 15/22; C07H 15/26; C11D 3/00; C11D 3/22; C11D 3/32

(52) U.S. Cl. CPC C07H 15/10; C07H 9/02; C07H 15/22; C07H 15/26; C11D 3/0026; C11D 3/221; C11D 3/32

(57) ABSTRACT

The present invention provides modified sophorolipids that can eliminate existing foam and/or prevent the formation of new foam. The modifications including peracetylation and/or global transformation of sophorose hydroxyl groups of sophorolipids to ethers to reduce the HLB value and create biobased defoamers with high biobased carbon content. The present invention also provides compositions comprising modified sophorolipids and methods of using such compositions for inhibiting and/or reducing foam formation.

### CROSS-REFERENCE TO RELATED APPLICATION

This application claims priority to U.S. Provisional Patent Application No. 63/388,419, filed Jul. 12, 2022, which is incorporated herein by reference in its entirety.

### BACKGROUND OF THE INVENTION

Foams are often produced as an unwanted consequence in the manufacture of various substances such as surfactants and proteins, particularly in processes involving significant shear forces near air-liquid interfaces, such as those involving aeration, pumping or agitation. In industrial processes, such as food processing, water treatment, pulp and paper, paint and coatings, and chemical manufacturing, foams pose serious problems. For example, they can cause defects on surface coatings, prevent the efficient filling of containers, and cause pump and sprayer malfunctions. Thus, defoamers are needed for such industrial processes.

Development of defoamers is typically to eliminate unnecessary foams in industrial production. Defoamers can come in many forms including hydrophobic solids, in-situ formation of hydrophobic solids, competition for surfactant, low surface tension liquids, and antagonistic surfactants or polymers. Commonly used defoamers include insoluble oils, polydimethylsiloxanes and other silicones, certain alcohols, stearates, and glycols.

Although defoamers must be able to help control or minimize the amount of foaming that occurs in the intended application, it is also important that defoamers do not have a negative impact on the functional properties of the materials with which they are used, or on the product quality. In certain target applications, removal of the defoamers may be required to reduce contaminations.

Because defoamers are usually hydrophobic, they may be difficult to sterilize, which can pose issues in, for example, the food and pharmaceutical industries. Thus, chemical defoamers are not always desired, especially in the food, feed and pharmaceutical industries. In addition, regulatory requirements in these industries limit the chemistries that are acceptable for use in antifoams and defoamers.

Biobased defoamers could provide excellent alternatives to current defoamers on the market. Thus, there is a need for developing biobased defoamers for use in various applications.

### BRIEF SUMMARY OF THE INVENTION

The subject invention provides modified sophorolipids having anti-foaming properties. The subject invention also provides compositions comprising such modified sophorolipids as anti-foaming agents. The subject invention further provides materials and methods for producing sophorolipids that are amenable to modification; and materials and methods for modifying sophorolipids to create biobased defoamers.

In one embodiment, the subject invention provides a compound that can be used as a foam control agent. In preferred embodiments compounds of the subject invention having anti-foaming properties, has a structure selected from:

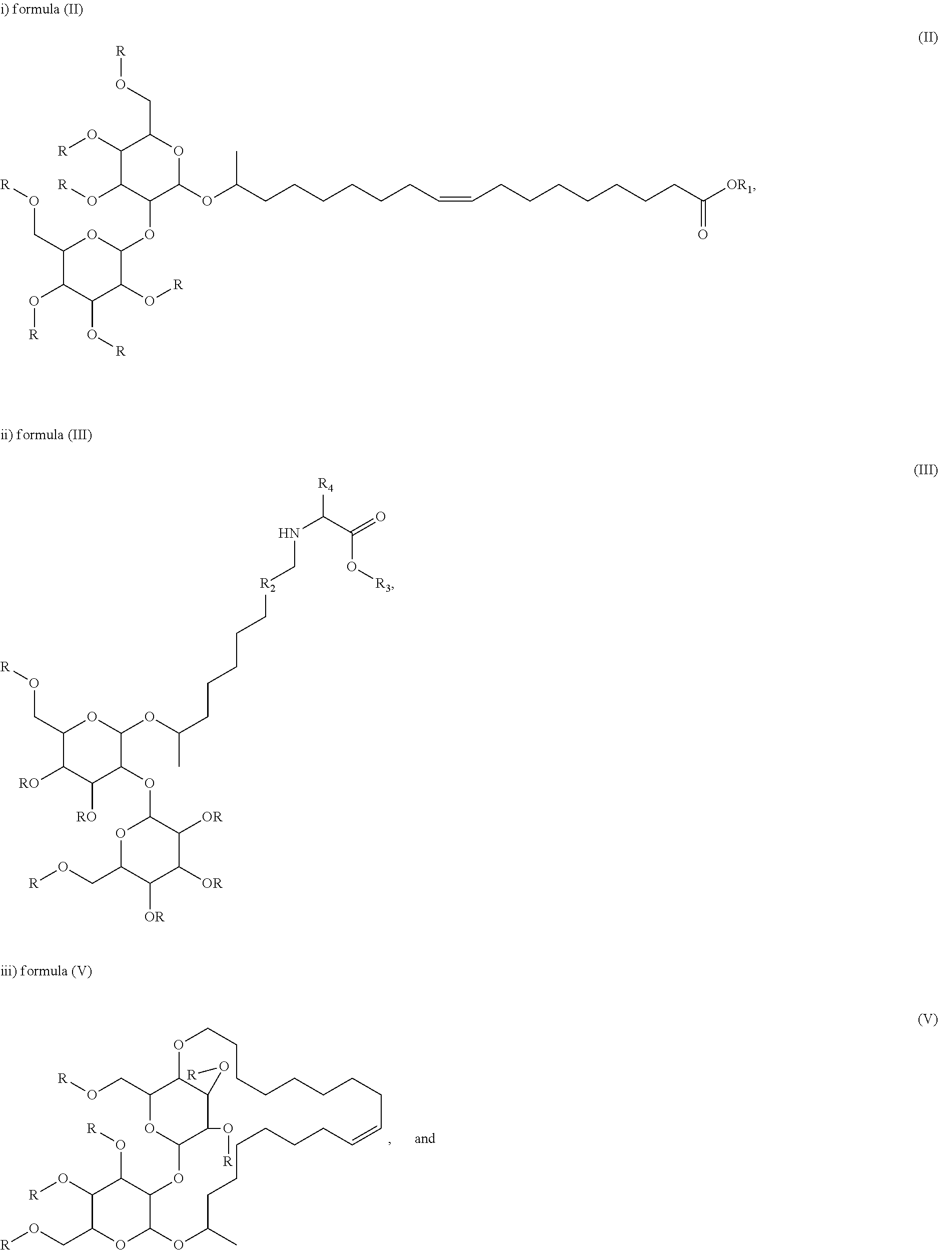

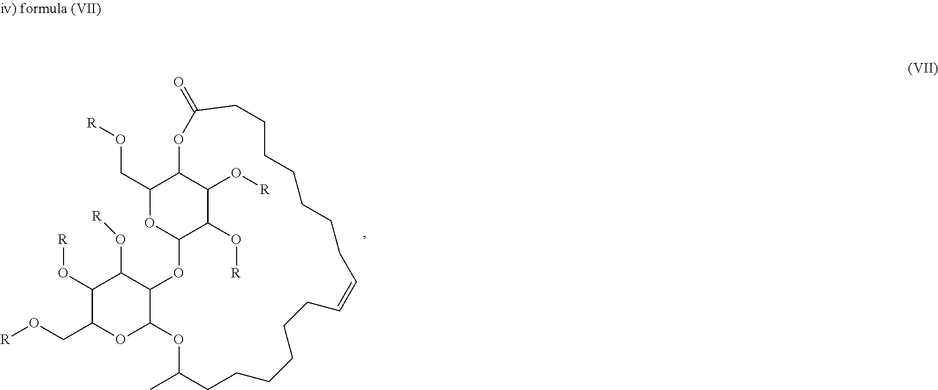

wherein each R is independently selected from hydrogen, alkyl, substituted alkyl, acyl, substituted acyl,

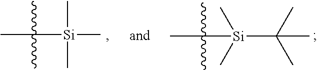

R₁ is selected from hydrogen, alkyl, substituted alkyl, heteroaryl, and substituted heteroaryl; R₂ is —(CH2)ₙ-, wherein n≥1; R₃ is selected from hydrogen, alkyl, substituted alkyl, heteroaryl, and substituted heteroaryl; and R₄ is selected from amino acid side chains, preferrably, those amino acid side chains with aromatics.

In preferred embodiments, each R is independently selected from H,

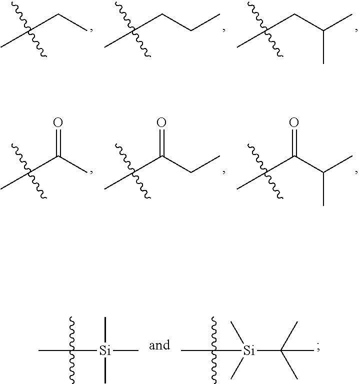

and R₁ is selected from hydrogen, methyl, ethyl, propyl, butyl, pentyl, i-propyl, i-butyl, and

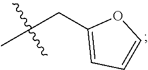 [SMILES: *Cc1ccco1]

R₂ is —(CH2)ₙ-, wherein 1≤n≤10; R₃ is selected from hydrogen, methyl, ethyl, propyl, butyl, pentyl, i-propyl, and i-butyl; and R₄ is selected from

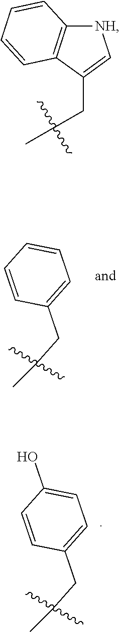 [SMILES: *Cc1c[nH]c2ccccc12 ; *Cc1ccccc1 ; *Cc1ccc(O)cc1]

More preferably, each R is independently selected from

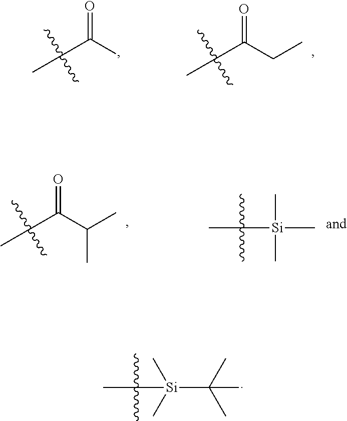

In a specific embodiment, the compound of the subject invention is selected from SH-global ether-COOH, SH-peracetylated-COOH, SH-peracetylated-furfuryl, SH-global ether-furfuryl, global ether lactonic, SH-peracetylated-OMe, SH-peracetylated-Oet, SH-peracetylated-O-i-Bu (isobutyl), SH-peracetylated-phenylalanine, and SH-peracetylated-tryptophan.

In one embodiment, the subject invention provides a defoaming composition comprising a compound of the subject invention, and optionally, one or more of the following defoaming compounds: silica, hydrophobed silica, polyethylene wax, ethylene bisstear amide wax, polypropylene wax, polydimethylsiloxane, organically modified polydimethylsiloxane, and hydrophobed polyethylene oxide glycerol ethers.

In one embodiment, the subject invention provides an anti-foaming detergent composition comprising the compound of the subject invention, and optionally, a detergent auxiliary component selected from enzymes, oxygen bleaching agents, bleaching activators, fluid reforming agents, and neutral inorganic salts.

In one embodiment, the subject invention provides a method for defoaming a liquid, the method comprising adding a defoaming composition of the subject invention to the liquid in need thereof, wherein the defoaming composition comprises an anti-foaming agent or foam control agent comprising one or more of the modified sophorolipids of the present invention.

In a specific embodiment, defoaming a liquid may include hindering the formation of foam or entrainment of gas in the liquid during preparation, vacuum stripping, sparging with a gas, or pumping of the liquid.

In a specific embodiment, the liquid may be selected from, for example, paints, fermentation broths, detergent compositions, crop protection and crop nutrient formulations, fiber finish and textile treatment formulations, drilling fluids, fracturing fluids, fluids used in pulp/paper processing, and cement compositions.

In one embodiment, the subject invention also provides a method for preventing or reducing foam formation in a liquid, wherein the method comprises combining the liquid with the defoaming composition of the subject invention. Preferably, the liquid is selected from paints, fermentation broths, detergent compositions, crop protection and crop nutrient formulations, fiber finish and textile treatment formulations, drilling fluids, fracturing fluids, and cement compositions.

### DETAILED DESCRIPTION

The subject invention provides biobased antifoaming agents or foam control agents, and compositions comprising the biobased antifoaming agent or foam control agent for industrial uses. Advantageously, the biobased antifoaming agent or foam control agent can eliminate existing foam and/or prevent the formation of further foam. Preferably, the biobased antifoaming agent or foam control agent is a modified sophorolipid that has reduced foaming properties.

The subject invention provides modified sophorolipids as defoamers for use in industrial processes, such as food processing, water treatment, pulp and paper processing, paints and coatings, and chemical manufacturing. The subject invention also provides defoamer compositions comprising a modified sophorolipid for use in various applications, for example, in agriculture, environmental protection, water treatment pharmaceutical industry, food processing, oil production, and the detergent industry.

The subject invention further provides materials and methods for producing sophorolipids that are amenable to modification; and materials and methods for modifying sophorolipids to create biobased defoamers.

Sophorolipid is one of the most promising and attractive biosurfactants that combines green chemistry with a low carbon footprint. Sophorolipids are glycolipid biosurfactants produced by, for example, various yeasts of the Starmerella clade. Biosurfactants are microbially-derived amphiphilic molecules consisting of both hydrophobic (e.g., a fatty acid) and hydrophilic domains (e.g., a sugar). Properties of biosurfactants, such as antimicrobial, emulsification and wettability, low toxicity, biodegradation and produced from renewable resources, allow them to be widely used in industrial production, agriculture, and daily life as wetting agents, emulsifiers, and detergents.

Sophorolipids are an amphiphilic molecule with an inherent hydrophobic half. Sophorolipids comprise a sophorose consisting of two glucose molecules, linked to a fatty acid by a glycosidic ether bond. Sophorolipids are categorized into two general forms: the lactonic form, where the carboxyl group in the fatty acid side chain and the sophorose moiety form a cyclic ester bond; and the acidic form, or linear form, where the ester bond is hydrolyzed.

Sophorolipids consist of a disaccharide sophorose linked to long chain hydroxy fatty acids. They can comprise a partially acetylated 2-O-β-D-glucopyranosyl-D-glucopyranose unit attached β-glycosidically to 17-L-hydroxyoctadecanoic or 17-L-hydroxy-A9-octadecenoic acid. The hydroxy fatty acid can have, for example, 11 to 20 carbon atoms, and may contain one or more unsaturated bonds.

Furthermore, the sophorose residue can be acetylated on the 6- and/or 6′-position(s). The fatty acid carboxyl group can be free (acidic or linear form) or internally esterified at the 4″-position (lactonic form). In most cases, fermentation of sophorolipids results in a mixture of hydrophobic (water-insoluble) sophorolipids, including, e.g., lactonic sophorolipids, mono-acetylated linear sophorolipids and di-acetylated linear sophorolipids, and hydrophilic (water-soluble) sophorolipids, including, e.g., non-acetylated linear sophorolipids.

Sophorolipids are frequently produced as a mixture of related molecules. Differences among the related molecules arise mainly from: their fatty acid structure (degree of unsaturation, chain length, position(s) of unsaturation and position of hydroxylation); whether they are produced in the linear or lactonic form; the acetylation pattern; the presence of stereoisomers; and/or whether the glycosidic bond on the fatty acid is at the ω-position (e.g., terminal) or ω−1 position (sub-terminal).

The sophorolipid compounds of the subject invention have many advantageous characteristics that make them superior to synthetic surfactants, such as biodegradability, low toxicity, high surface and interfacial activities, and stability under wide ranges of temperatures, pressures, and ionic strengths. On the other hand, functional properties of sophorolipids can differ between lactonic and acidic forms. For example, acidic sophorolipids generally have higher hydrophile-lipophile balance (HLB) than lactonic sophorolipids, while lactonic sophorolipids generally have lower HLB and greater surface tension-reducing properties than acidic sophorolipids. Additionally, acidic sophorolipids are typically highly water soluble due to their free carboxylic acid groups.

As used herein, the term “sophorolipid,” and “sophorolipid molecule” includes all forms, and isomers thereof, of sophorolipid molecules, including, for example, acidic (linear) sophorolipids and lactonic sophorolipids. Further included are mono-acetylated sophorolipids, di-acetylated sophorolipids, esterified sophorolipids, sophorolipids with varying hydrophobic chain lengths, sophorolipids with fatty acid-amino acid complexes attached, and other, including those that are and/or are not described within in this disclosure.

### Modified Sophorolipids

Linear sophorolipid molecules can be represented by general formula (I):

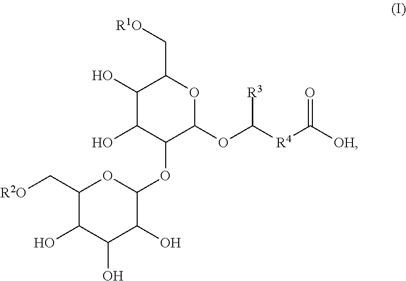

wherein R¹ and R² are each independently selected from hydrogen, alkyl, substituted alkyl, acyl, substituted acyl, alkenyl, and substituted alkenyl; R³ is hydrogen or alkyl; and R⁴ is alkane, substituted alkane, alkylene, substituted alkylene, alkenylene or substituted alkenylene. Preferrably, R¹ and R² are hydrogen; and R³ is methyl.

Sophorolipid molecules can be obtained as a collection of 30 or more molecules having different fatty acid chain lengths and degrees of unsaturation (R⁴), and, in some instances, having an acetylation or protonation at R¹ and/or R².

Examples of the substituents include halogen atoms, hydroxyl, lower (C1-6) alkyl groups, halo lower (C1-6) alkyl groups, hydroxy lower (C1-6) alkyl groups, halo lower (C1-6) alkoxy groups, and others. R⁴ typically has 11 to 20 carbon atoms. In preferred embodiments of the subject invention, R⁴ has 16-17 carbon atoms.

In one embodiment, the subject invention provides modified sophorolipids, in which a sophorolipid or a truncated sophorolipid is linked to an amino acid via a primary amine. By “truncated,” it is meant that the carbon chain of the fatty acid is shorter than the carbon chain of the fatty acids normally present in sophorolipids.

In one embodiment, the present invention provides modified sophorolipids. Preferably, the modification includes, for example, peracetylation and esterification. In preferred embodiments, the modified sophorolipid of the subject invention has a general structure of formula (II):

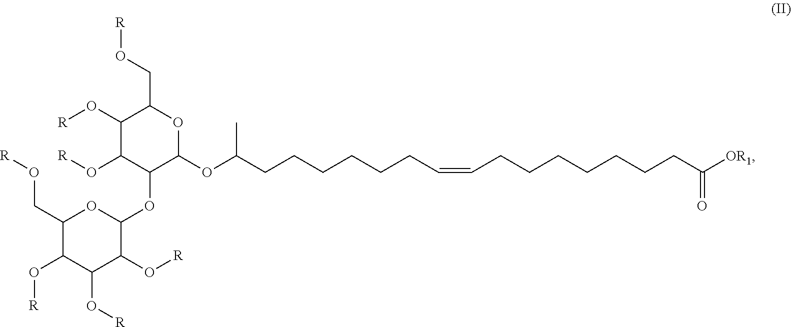

wherein each R is independently selected from hydrogen, alkyl, substituted alkyl, acyl, substituted acyl,

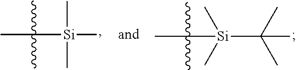

and R₁ is selected from hydrogen, alkyl, substituted alkyl, heteroaryl, and substituted heteroaryl. Preferably, each R is independently selected from

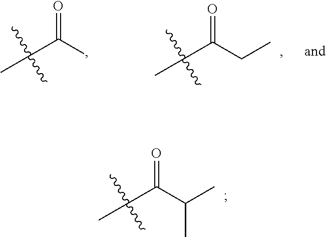

and; and R₁ is selected from hydrogen, methyl, ethyl, propyl, butyl, pentyl, i-propyl, i-butyl, and

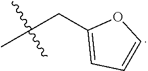 [SMILES: *Cc1ccco1]

In one embodiment, the present invention provides modified sophorolipids having a general structure of formula (III):

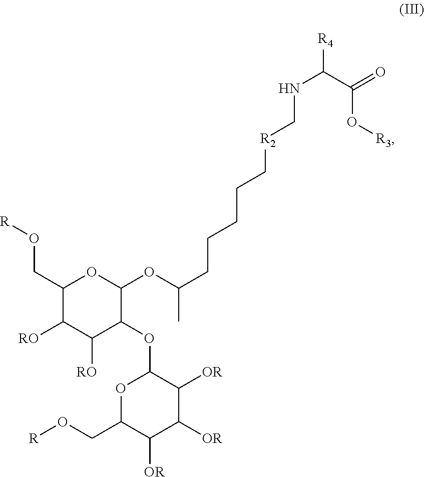

wherein each R is independently selected from hydrogen, alkyl, substituted alkyl, acyl, substituted acyl,

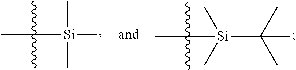

R₂ is —(CH2)ₙ-, wherein n≥1; R₃ is selected from hydrogen, alkyl, substituted alkyl, heteroaryl, and substituted heteroaryl; and R₄ is selected from amino acid side chains, preferrably, those with aromatics, for example,

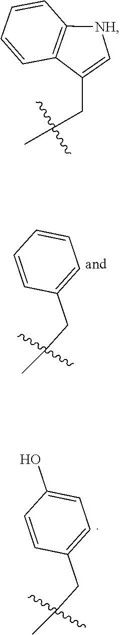 [SMILES: *Cc1c[nH]c2ccccc12 ; *Cc1ccccc1 ; *Cc1ccc(O)cc1]

Preferably, each R is independently selected from,

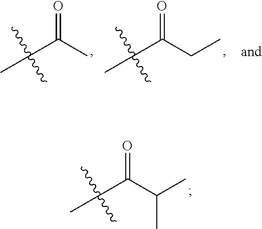

1≤n≤10; and R₃ is selected from hydrogen, methyl, ethyl, propyl, butyl, pentyl, propyl, i-butyl, and

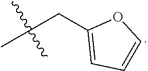 [SMILES: *Cc1ccco1]

In one embodiment, the present invention provides modified sophorolipids having the general structure of formula (IV), which are cyclic ether analogs of lactonic sophorolipids. Thus, the present invention provides compounds of the formula (IV):

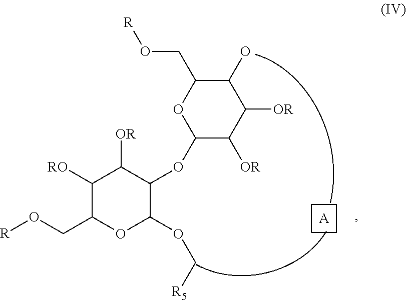

wherein each R is independently selected from hydrogen, alkyl, substituted alkyl, acyl, substituted acyl,

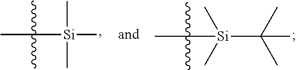

R₅ is hydrogen or alkyl; and A is a saturated or unsaturated aliphatic chain that is optionally substituted. Preferably, each R is independently selected from H,

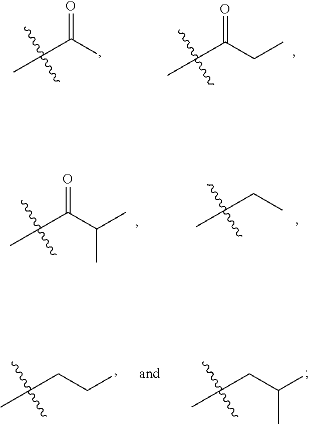

and R₅ is hydrogen or methyl.

In preferred embodiments, the aliphatic chain A has 10 to 21 carbons such that the total number of carbons in the aliphatic chain A, R₅, and the carbon to which R₅ is attached is 12 to 22 carbons.

In some embodiments, R is ethyl, R₅ is methyl, and A is an unsaturated aliphatic chain having 16 carbons with one, two, or three unsaturation.

In some embodiments, R is ethyl, R₅ is hydrogen, and A is an unsaturated aliphatic chain having 17 carbons with one, two, or three unsaturation.

In some embodiments, R is ethyl, R₅ is hydrogen, and A is an unsaturated aliphatic chain having 16 carbons with one, two, or three unsaturation.

In some embodiments, R is ethyl, R₅ is methyl, and A is an unsaturated aliphatic chain having 17 carbons with one, two, or three unsaturation.

In some embodiments, R is hydrogen, R₅ is methyl, and A is an unsaturated aliphatic chain having 16 carbons with one, two, or three unsaturation.

In some embodiments, R is hydrogen, R₅ is hydrogen, and A is an unsaturated aliphatic chain having 17 carbons with one, two, or three unsaturation.

In other embodiments, each R is independently hydrogen or ethyl, R₅ is methyl, and A is an unsaturated aliphatic chain having 16 carbons with one unsaturation.

In other embodiments, each R is independently hydrogen or ethyl, R₅ is methyl, and A is an unsaturated aliphatic chain having 17 carbons with one unsaturation.

In other embodiments, each R is independently hydrogen or ethyl, R₅ is methyl, and A is a fully saturated aliphatic chain having 16 carbons.

In other embodiments, each R is independently hydrogen or ethyl, R₅ is hydrogen, and A is a fully saturated aliphatic chain having 17 carbons.

In one embodiment, the present invention provides modified sophorolipids having the general formula (V):

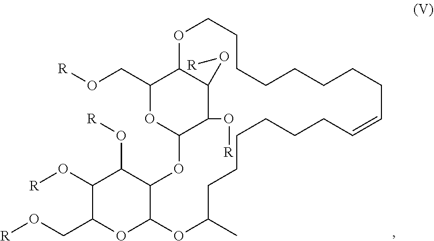

wherein each R is independently selected from hydrogen, alkyl, substituted alkyl, acyl, substituted acyl,

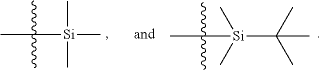

Preferably, each R is independently selected from H,

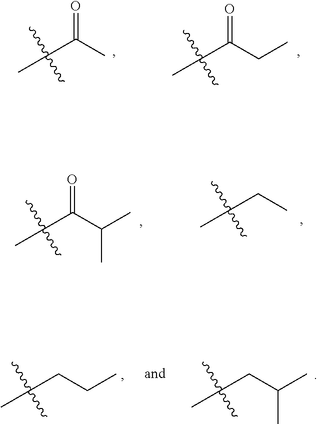

In one embodiment, the present invention provides modified sophorolipids having the general formula (VI):

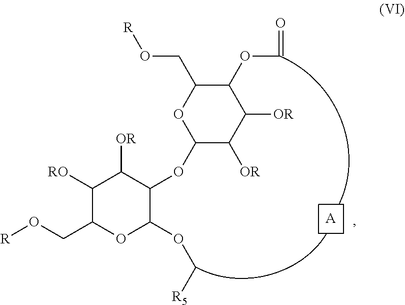

wherein each R is independently selected from hydrogen, alkyl, substituted alkyl, acyl, substituted acyl,

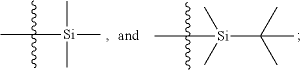

R₅ is hydrogen or alkyl; and A is a saturated or unsaturated aliphatic chain that is optionally substituted. Preferably, each R is independently selected from H,

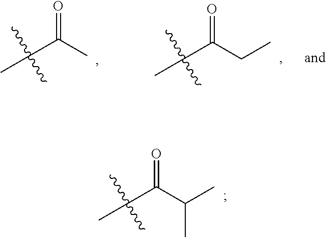

and R5 is hydrogen or methyl.

In preferred embodiments, the aliphatic chain A has 10 to 21 carbons such that the total number of carbons in the aliphatic chain A, R₅, and the carbon to which R₅ is attached is 12 to 22 carbons.

In some embodiments, R is ethyl, R₅ is methyl, and A is an unsaturated aliphatic chain having 16 carbons with one, two, or three unsaturation.

In some embodiments, R is ethyl, R₅ is hydrogen, and A is an unsaturated aliphatic chain having 17 carbons with one, two, or three unsaturation.

In some embodiments, R is ethyl, R₅ is hydrogen, and A is an unsaturated aliphatic chain having 16 carbons with one, two, or three unsaturation.

In some embodiments, R is ethyl, R₅ is methyl, and A is an unsaturated aliphatic chain having 17 carbons with one, two, or three unsaturation.

In some embodiments, R is hydrogen, R₅ is methyl, and A is an unsaturated aliphatic chain having 16 carbons with one, two, or three unsaturation.

In some embodiments, R is hydrogen, R₅ is hydrogen, and A is an unsaturated aliphatic chain having 17 carbons with one, two, or three unsaturation.

In other embodiments, each R is independently hydrogen or ethyl, R₅ is methyl, and A is an unsaturated aliphatic chain having 16 carbons with one unsaturation.

In other embodiments, each R is independently hydrogen or ethyl, R₅ is methyl, and A is an unsaturated aliphatic chain having 17 carbons with one unsaturation.

In other embodiments, each R is independently hydrogen or ethyl, R₅ is methyl, and A is a fully saturated aliphatic chain having 16 carbons.

In other embodiments, each R is independently hydrogen or ethyl, R₅ is hydrogen, and A is a fully saturated aliphatic chain having 17 carbons.

In one embodiment, the present invention provides modified sophorolipids having the general formula (VII):

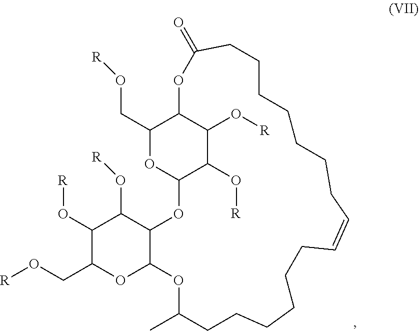

wherein each R is independently selected from hydrogen, alkyl, substituted alkyl, acyl, substituted acyl,

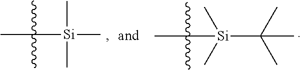

Preferably, each R is independently selected from H,

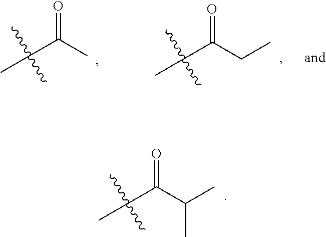

In one embodiment, the modified sophorolipids of the present invention have a reduced HLB value compared to the linear or lactonic sophorolipids. In specific embodiments, the modified sophorolipids of the present invention have a HLB value equal to or less than 6, preferably, equal to or less than 5, more preferably, equal to or less than 4, most preferably, equal to or less than 3.

In a specific embodiment, the modified sophorolipid is selected from SH-global ether-COOH, SH-peracetylated-COOH, SH-peracetylated-furfuryl, SH-global ether-furfuryl, global ether lactonic, SH-peracetylated-OMe, SH-peracetylated-Oet, SH-peracetylated-O-i-Bu (isobutyl), SH-peracetylated-phenylalanine, and SH-peracetylated-tryptophan.

### Selected Definitions

As used herein, the term “foam” refers to a dispersion of gas bubbles in or on a liquid, in a gel or in a semisolid. The gas bubbles may be dispersed throughout the liquid phase in a heterogeneous or homogeneous manner. Illustrative examples of foams include gases such as air, nitrogen, oxygen, helium or hydrogen entrapped in a liquid such as water or an oil. A foam may be transient, unstable or stable.

As used herein, “surfactant” refers to a substance or compound that reduces surface tension when dissolved in water or water solutions, or that reduces interfacial tension between two liquids, or between a liquid and a solid. The term “surfactant” thus includes cationic, anionic, nonionic, zwitterionic, amphoteric agents and/or combinations thereof. By “biosurfactant” is meant a surface active agent produced by a living organism, and/or produced using naturally-derived substrates. “Surfactant” and “biosurfactant” also pertain to substances that cause foaming.

As used herein, the term “alkyl” refers to straight chain or branched hydrocarbon groups. Suitable alkyl groups include, but are not limited to methyl, ethyl, propyl, isopropyl, butyl, isobutyl, pentyl, hexyl, heptyl, octyl, nonyl, decyl, dodecyl, tetradecyl, hexadecyl and octadecyl. The term alkyl may be prefixed by a specified number of carbon atoms to indicate the number of carbon atoms or a range of numbers of carbon atoms that may be present in the alkyl group such as C1-C10 alkyl, C1-C20 alkyl, and C10-C20 alkyl. For example, C1-C3 alkyl refers to methyl, ethyl, propyl and isopropyl.

As used herein, an “isolated” or “purified” nucleic acid molecule, polynucleotide, polypeptide, protein or organic compound, such as a small molecule, is substantially free of other compounds, such as cellular material, with which it is associated in nature. A purified or isolated polynucleotide (ribonucleic acid (RNA) or deoxyribonucleic acid (DNA)) is free of the genes or sequences that flank it in its naturally-occurring state. A purified or isolated polypeptide is free of other molecules, or the amino acids that flank it, in its naturally-occurring state. An “isolated” strain means that the strain is removed from the environment in which it exists in nature. Thus, the isolated strain may exist as, for example, a biologically pure culture, or as spores (or other forms of the strain).

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19 and 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein “fermentation” refers to growth or cultivation of cells under controlled conditions. The growth could be aerobic or anaerobic. Unless the context requires otherwise, the phrase is intended to encompass both the growth phase and product biosynthesis phase of the process.

As used herein, a “broth,” “culture broth,” or “fermentation broth” refers to a culture medium comprising at least nutrients. If the broth is referred to after a fermentation process, the broth may comprise microbial growth byproducts and/or microbial cells as well.

The microbe growth vessel used according to the subject invention can be any fermenter or cultivation reactor for industrial use. As used herein, the term “reactor,” “bioreactor,” “fermentation reactor” or “fermentation vessel” includes a fermentation device consisting of one or more vessels and/or towers or piping arrangements. Examples of such reactor includes, but are not limited to, the Continuous Stirred Tank Reactor (CSTR), Immobilized Cell Reactor (ICR), Trickle Bed Reactor (TBR), Bubble Column, Gas Lift Fermenter, Static Mixer, or other vessel or other device suitable for gas-liquid contact. In some embodiments, the bioreactor may comprise a first growth reactor and a second fermentation reactor. As such, when referring to the addition of substrate to the bioreactor or fermentation reaction, it should be understood to include addition to either or both of these reactors where appropriate.

As used herein, “reduces” means a negative alteration, and “increases” means a positive alteration, wherein the alteration is at least 0.001%, 0.01%, 0.1%, 1%, 5%, 10%, 15%, 20%, 25%, 30%, 35%, 45%, 50%, 55%, 60%, 65%, 70%, 75%, 80%, 85%, 90%, 95%, 99%, or 100%, inclusive of all values therebetween.

The term “subject” or “patient,” as used herein, describes an organism, including mammals such as primates. Mammalian species that can benefit from the disclosed methods of treatment include, but are not limited to, apes, chimpanzees, orangutans, humans, and monkeys; domesticated animals such as dogs, cats; live stocks such as horses, cattle, pigs, sheep, goats, and chickens; and other animals such as mice, rats, guinea pigs, and hamsters.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially” of the recited component(s).

Unless specifically stated or obvious from context, as used herein, the term “or” is understood to be inclusive. Unless specifically stated or obvious from context, as used herein, the terms “a,” “an” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof. All references cited herein are hereby incorporated by reference.

### Production of Sophorolipids

The subject invention provides materials and methods for producing and modifying sophorolipids. Advantageously, the subject invention is suitable for industrial scale production of purified sophorolipids and uses safe and environmentally-friendly materials and processes.

Sophorolipids are generally obtained from fermentation by microorganisms that use as carbon sources pure fatty acids, fatty acid mixtures, pure fatty acid esters, mixtures of fatty acid esters, triglycerides along with carbohydrate sources such as corn syrup, dextrins and glucose using a fermentation process comprising a wild-type or engineered microorganism. The production of sophorolipids with the use of renewable substrates and different microbial species, as well as the variation in culture parameters (e.g., incubation time, stirring speed, pH of the medium and added nutrients), allow for the acquisition of compounds with distinct structural and physical properties. This makes it possible to produce a wide variety of compounds that exhibit different physical, chemical, biochemical, and biophysical properties.

In preferred embodiments of the subject invention, the subject methods initially comprise producing standard sophorolipid molecules that are then used for producing modified sophorolipids. In certain embodiments, this entails cultivating a sophorolipid-producing yeast in a submerged fermentation reactor comprising a tailored oleochemical feedstock to produce a yeast culture product, said yeast culture product comprising fermentation broth, yeast cells and sophorolipids having a mixture of two or more molecular structures.

The mixture of molecular structures can comprise, for example, lactonic sophorolipids, linear sophorolipids, de-acetylated sophorolipids, mono-acetylated sophorolipids, di-acetylated sophorolipids, esterified sophorolipids, sophorolipids with varying hydrophobic chain lengths, sophorolipids with fatty acid-amino acid complexes attached, and others, including those that are and/or are not described within in this disclosure.

In certain embodiments, the distribution of the mixture of sophorolipids molecules can be altered by adjusting fennentation parameters, such as, for example, feedstock, fermentation time, and dissolved oxygen levels.

The sophorolipids according to the present invention can be derived via a fermentation process from a recombinant organism or by a strain that naturally produces sophorolipids. Non-limiting examples of sophorolipid-producing organisms include Candida bombicola, Candida apicola, Candida bogoriensis, Yarrowia lipolytica, Starmerella bombicola, Stannerella clade, Rhodolorula bogoriensis, Wickerhamiella domericqiae, and Wickerhamomyces anomalus. Some recombinant sophorolipid-producing microbes have been reported to allow control of sophorolipid structure. As a non-limiting example, certain recombinant S. bombicola strains may be utilized to produce either solely lactonic or solely acidic sophorolipids. In addition, a recombinant Candida bombicola strain with an acetyltransferase gene knockout can be used to produce sophorolipids without acetylation.

In preferred embodiments, the microorganism is a yeast or fungus. Examples of yeast and fungus species suitable for use according to the current invention, include, but are not limited to, Acaulospora, Aspergillus, Aureobasidium (e.g., A. pullulans), Blakeslea, Candida (e.g., C. albicans, C. apicola), Cryptococcus, Debaryomyces (e.g., D. hansenii), Entomophthora, Fusarium, Hanseniaspora (e.g., H. uvarum), Hansenula, Issatchenkia, Kluyveromyces, Mortierella, Mucor (e.g., M. piriformis), Meyerozyma (e.g., M. guilliermondii), Penicillium, Phythium, Phycomyces, Pichia (e.g., P. anomala, P. guilliermondii, P. occidentalis, P. kudriavzevii), Pseudozyma (e.g., P. aphidis), Rhizopus, Saccharomyces (S. cerevisiae, S. boulardii sequela, S. torula), Starmerella (e.g., S. bombicola), Torulopsis, Thraustochytrium, Trichoderma (e.g., T. reesei, T. harzianum, T virens), Ustilago (e.g., U. maydis), Wickerhamomyces (e.g., W. anomalus), Williopsis, and Zygosaccharomyces (e.g., Z. bailii).

In preferred embodiments, the microorganisms are selected from, for example, Starmerella spp. yeasta and/or Candida spp. yeasta, e.g., Starmerella (Candida) bombicola, Candida apicola, Candida batistae, Candida floricola, Candida riodocensis, Candida stellate and Candida kuoi. In a specific embodiment, the microorganism is Starmerella bombicola, e.g., strain ATCC 22214.

In one embodiment, the fermentation reactor may have functional controls/sensors or may be connected to functional controls/sensors to measure important factors in the cultivation process, such as pH, oxygen, pressure, temperature, agitator shaft power, humidity, viscosity and/or microbial density and/or metabolite concentration.

In a further embodiment, the vessel may also be able to monitor the growth of microorganisms inside the vessel (e.g., measurement of cell number and growth phases). Alternatively, samples may be taken from the vessel for enumeration, purity measurements, sophorolipid concentration, and/or visible oil level monitoring. For example, in one embodiment, sampling can occur every 24 hours.

The microbial inoculant according to the subject methods preferably comprises cells and/or propagules of the desired microorganism, which can be prepared using any known fermentation method. The inoculant can be pre-mixed with water and/or a liquid growth medium, if desired.

The microorganisms utilized according to the subject invention may be natural, or genetically modified microorganisms. For example, the microorganisms may be transformed with specific genes to exhibit specific characteristics. The microorganisms may also be mutants of a desired strain.

In one embodiment, a single type of microorganism is grown in a reactor system. In alternative embodiments, multiple microorganisms, which can be grown together without deleterious effects on growth or the resulting product, can be grown in a single reactor system. There may be, for example, 2 to 3 or more different microorganisms grown in a single reactor at the same time. In some embodiments, more than one microorganism grows symbiotically in the reactor.

In certain embodiments, the cultivation may be supplemented with a carbon source. The carbon source can be a carbohydrate, such as glucose, dextrose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as canola oil, soybean oil, rice bran oil, olive oil, corn oil, sunflower oil, sesame oil, and/or linseed oil; powdered molasses, etc. These carbon sources may be used independently or in a combination of two or more.

In certain embodiments, the cultivation may be supplemented with high-oleic acid and/or exclusively-oleic acid oleochemical feedstock, which results in a yeast culture product comprising a narrower diversity of sophorolipid molecular structures than with feedstocks containing sources of other fatty acids, wherein the principal sophorolipid molecules produced contain an 18C carbon chain and a single unsaturated bond at the ninth carbon.

In one embodiment, the liquid growth medium comprises a nitrogen source. The nitrogen source can be, for example, yeast extract, potassium nitrate, ammonium nitrate, ammonium sulfate, ammonium phosphate, ammonia, urea, and/or ammonium chloride. These nitrogen sources may be used independently or in a combination of two or more.

In one embodiment, one or more inorganic salts may also be included in the liquid growth medium. Inorganic salts can include, for example, potassium dihydrogen phosphate, monopotassium phosphate, dipotassium hydrogen phosphate, disodium hydrogen phosphate, potassium chloride, magnesium sulfate, magnesium chloride, iron sulfate, iron chloride, manganese sulfate, manganese chloride, zinc sulfate, lead chloride, copper sulfate, calcium chloride, calcium carbonate, calcium nitrate, magnesium sulfate, sodium phosphate, sodium chloride, and/or sodium carbonate. These inorganic salts may be used independently or in a combination of two or more.

In one embodiment, growth factors and trace nutrients for microorganisms are included in the medium. Inorganic nutrients, including trace elements such as iron, zinc, copper, manganese, molybdenum and/or cobalt may also be included in the medium. Furthermore, sources of vitamins, essential amino acids, proteins and microelements can be included, for example, corn flour, peptone, yeast extract, potato extract, beef extract, soybean extract, banana peel extract, and the like, or in purified forms. Amino acids such as, for example, those useful for biosynthesis of proteins, can also be included.

The method of cultivation can further provide oxygenation to the growing culture. One embodiment utilizes slow motion of air to remove low-oxygen containing air and introduce oxygenated air. The oxygenated air may be ambient air supplemented daily through mechanisms including impellers for mechanical agitation of the liquid, and air spargers for supplying bubbles of gas to the liquid for dissolution of oxygen into the liquid.

In one embodiment, the microorganisms can be grown on a solid or semi-solid substrate, such as, for example, corn, wheat, soybean, chickpeas, beans, oatmeal, pasta, rice, and/or flours or meals of any of these or other similar substances.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, calcium carbonate, sodium chloride, and/or sodium carbonate. These inorganic salts may be used independently or in a combination of two or more.

In some embodiments, when, for example, the microbes used to inoculate the substrate are in spore form (e.g., bacterial endospores), germination enhancers can be added to the substrate. Examples of germination enhancers according to the present invention include, but are not limited to, L-alanine, manganese, L-valine, and L-asparagine or any other known germination enhancer.

The pH of the culture should be suitable for the microorganism of interest. Buffers, and pH regulators, such as carbonates and phosphates, may be used to stabilize pH near a preferred value. In certain embodiments, a base solution is used to adjust the pH of the culture to a favorable level, for example, a 15% to 30%, or a 20% to 25% NaOH solution. The base solution can be included in the growth medium and/or it can be fed into the fermentation reactor during cultivation to adjust the pH as needed. When metal ions are present in high concentrations, use of a chelating agent in the liquid medium may be necessary.

The method and equipment for cultivation of microorganisms and production of the microbial by-products can be performed in a batch, quasi-continuous, or continuous processes.

In one embodiment, the method of cultivation is carried out at about 5° to about 1000 C, about 150 to about 600 C, about 200 to about 450 C, about 220 to about 35° C., or about 24° to about 28° C. In one embodiment, the cultivation may be carried out continuously at a constant temperature. In another embodiment, the cultivation may be subject to changing temperatures.

According to the subject methods, the microorganisms can be cultivated in the fermentation system for a time period sufficient to achieve a desired effect, e.g., production of a desired amount of cell biomass or a desired amount of sophorolipids.

In certain embodiments, fermentation of the yeast culture occurs for about 40 to 150 hours, or about 48 to 140 hours, or about 72 to 130 hours or about 96 to 120 hours. In certain specific embodiments, fermentation time ranges from 48 to 72 hours, or from 96 to 120 hours.

In some embodiments, the sophorolipids produced by microorganisms of interest may be retained in the microorganisms or secreted into their growth medium. The sophorolipid content can be, for example, at least 20%, 30%, 40%, 50%, 60%, 70%, 80%, or 90%.

The growth medium may contain compounds that stabilize the activity of the sophorolipids. The sophorolipids can be purified, or the sophorolipids can be used in crude form, meaning they are not separated from the fermentation broth in which they were produced.

In certain embodiments, the sophorolipid is isolated and/or purified from the growth medium resulting from fermentation of a biosurfactant-producing microorganism. Isolation and purification can be easily achieved using standard methods or techniques described in the literature. The sophorolipid can be further concentrated, if desired.

### Modification of Sophorolipids

In one embodiment, the subject invention provides a synthetic procedure to create a sophorolipid scaffold to add modifications to the fatty acid end for enhancing anti-foaming ability. “Scaffold” is a starting point for creating a structure-activity relationship, e.g., determining how each small modification contributes to defoaming capabilities. For example, transforming the carboxylic acid into a methyl, ethyl, or isobutyl ester can be used to create anti-foaming agents out of fatty acid such as stearic acid.

In some embodiments, the modified sophorolipids according to the present invention are obtained by chemically modifying sophorolipids that have been obtained from the product of fermentation by sophorolipid-producing microbes. The sophorolipids to be chemically modified may be obtained by fermentation methods and processes as described above and/or any other methods known in the art. Any suitable techniques and chemical reactions known in the art may be used to modify sophorolipids.

Chemical modifications can also be made, for example, to alter the degree of unsaturation on the fatty acid chain. Commonly known hydrogenation or dehydrogenation reactions, or addition or elimination reactions may be used.

Sophorolipids containing an unsaturated bond at a specific position allows for site-directed functionalization of the sophorolipid molecule. In certain embodiments, the linear sophorolipids are ozonated with ozone gas. During ozonolysis of the linear sophorolipids, the olefin moiety of the sophorolipid molecule is converted to an ozonide, a reactive 5-membered ring. In preferred embodiments, the sophorolipid containing the ozonide is reduced to afford an aldehyde handle. In a specific embodiment, the reducing agent is triphenyl phosphine used in equimolar concentrations to the sophorolipid-ozonide.

In certain embodiments, the reactive aldehyde handle, whether produced via ozonolysis or oxidative cleavage, is then used as the site for addition of, for example, a primary amine via reductive amination. In certain embodiments, the reductive amination comprises introducing a primary amine to the sophorolipid-aldehyde under reducing conditions. This produces a stable secondary amine that serves as a covalent linkage between the sophorolipid scaffold and the cargo (e.g., amino acids) of the primary amine.

In certain embodiments, the reducing agent is sodium cyanoborohydride, sodium triacetoxyborohydride or sodium borohydride. In some embodiments, the linear sophorolipid aldehyde is extracted with ethyl acetate from the aqueous mixture and concentrated and dried under reduced pressure (e.g., about 200 to 250 mbar, or about 240 mbar) at a temperature of about 35 to 45° C. The dried crude linear sophorolipid aldehyde can then be dissolved in a reaction medium comprising tetrahydrofuran (THF) and water. The percentage of water used as the reaction medium preferably does not exceed 50% water, and typically is from 0 to 25%.

In some embodiments, the amide coupling addition of amino acids such as phenylalanine methyl ester and tryptophan methyl ester could retain the biobased carbons of the amino acids while adding aromatic rings that keep the HLB of sophorolipid in the anti-foaming range. Biobased carbons are carbons directly derived from, for example, agricultural sources, e.g., sugar-based fermentation products. Biobased carbons within a molecule are apart from the molecule directly because of biological processes. Biobased carbons can be quantitatively measured by C14 radiocarbon testing.

In one embodiment, modified sophorolipids of the present invention incorporate within their structure an amino acid linked to the fatty acid chain of the sophorolipid structure via the primary amine. By covalently attaching an amino acid to a sophorolipid or a truncated sophorolipid, the modified sophorolipids exhibit anti-foaming capabilities. Further, the addition of amino acids contributes biobased carbons instead of synthetic carbons, increasing the biobased carbon % in the modified sophorolipids, which enhances the defoaming properties of the modified sophorolipids.

Biobased carbon % represents the number of biobased carbon out of the total number of atoms within a molecule (e.g., biobased carbon %=(the number of biobased carbons/total number of atoms)×100).

Amino acid as used herein has at least one amino group and at least one acid such as carboxyl group in its backbone, along with a side chain that is different for each amino acid. The side chain can be a variety of substituents, including but are not limited to, hydrogen (e.g., as in glycine), organic, semi-organic or inorganic, and a ring that is cyclized on to the amine (e.g., as in proline).

The amino acid as used herein may be L-amino acid or D-amino acid. It can be natural amino acids (such as essential amino acids, non-essential amino acids, or conditional amino acids) or synthetic/modified amino acids that do not exist in nature. The present invention encompasses amino acids that are both incorporated into proteins and not incorporated into proteins. Amino acids that exist in nature may be purified products or can be artificially synthesized.

In some embodiments, the amino acid according to the present invention is an alpha (α)-amino acid, which contains an amino group and a carboxyl group attached to the same carbon adjacent to the carboxyl group designated as the α-carbon. Non-limiting examples of α-amino acids include, for example, glycine, methionine, lysine, phenylalanine, tyrosine, tryptophan, and glutamine.

Other non-limiting examples of amino acids that can be used in the present invention include, but are not limited to, alanine, ß-alanine, arginine, asparagine, aspartic acid, cysteine, homocysteine, glutamic acid, histidine, homotaurine, isoleucine, leucine, phenylalanine, proline, serine, threonine, tryptophan, tyrosine, valine, theanine, D-2-amino-3-guanidinopropionic acid, GABA, citrulline, tranexamic acid, aminocaproic acid, 4-amino-5-hexenoic acid, 4-oxaproline, 4-thioproline, 2-azaproline, 4-hydroxyproline, 1,5-disubstituted tetrazole, 2-amino isobutyric acid, sarcosine, 1-aminocyclopentane-1-carboxylic acid, beta alanine, 2-amino-cyclopentane carboxylic acid (beta-proline), 5-hydroxylysine, hydroxylysine-5-sulfate, hydroxylysine-5-nitrate, hydroxylysine-5-phosphate, serine-3-sulfate, threonine-3-sulfate, serine-3-nitrate, threonine-3-nitrate, serine-3-phosphate, threonine-3-phosphate, 2-hydroxy alkanoic acid, 5-aminopentanoic acid, and 5-amino-2-oxopentanoic acid.

In some embodiments, the modified sophorolipids having an attached amino acid can further be esterified by a functional group via the carboxyl group of the amino acid. The functional groups include, but are not limited to, for example, methyl, ethyl, propyl, butyl, pentyl, i-propyl, i-butyl, and

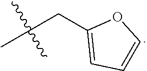 [SMILES: *Cc1ccco1]

Lactonic species inherently produce lower HLB values, so peracetylation or global transformation of sophorose hydroxyl groups to ethers would also lower the HLB value, creating biobased anti-foaming agents with high percentage of carbon (i.e., carbon %).

In one embodiment, the modified sophorolipids of the subject invention have a biobased carbon content, for example, from about 50% to about 100%, about 50% to about 95%, about 50% to about 90%, about 50% to about 80%, about 50% to about 70%, about 50% to about 60%, about 55% to about 90%, about 55% to about 85%, about 55% to about 80%, about 55% to about 75%, about 60% to about 85%, about 60% to about 80%, about 60% to about 75%, or about 60% to about 70%.

One embodiment is directed to a method for producing the modified sophorolipids comprising: obtaining the linear sophorolipid molecules; and functionalizing the linear sophorolipid molecules by converting all, or a majority, of the linear sophorolipid molecules to a modified sophorolipid molecule.

### Compositions and Uses Thereof

The subject invention provides compositions comprising biobased antifoaming agents or foam control agents of the subject invention for industrial uses. The biobased antifoaming agents or foam control agents eliminate existing foam and prevent the formation of further foam. Advantageously, in preferred embodiments, the biobased antifoaming agents or foam control agents are biodegradable and do not need to be removed from materials, with which they are used, or the final products.

In one embodiment, the subject invention provides a defoamer composition comprising the modified sophorolipid of the subject invention. In one embodiment, the subject invention provides an anti-foaming detergent composition comprising the modified sophorolipid of the subject invention. In one embodiment, the subject invention provides a cleaning composition comprising the modified sophorolipid of the subject invention. In one embodiment, the subject invention provides a coating composition, preferably, a paint, comprising the modified sophorolipid of the subject invention. Advantageously, the present invention can be used without causing harm to users and without releasing large quantities of polluting and toxic compounds into the environment.

In further embodiments, a modified sophorolipid of the subject invention can be used in pulp and paper processing.

In some embodiments, the composition of the subject invention comprises, the modified sophorolipid, for example, at least, 0.001%, 0.005%, 0.01%, 0.05%, 0.1%, 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, or 70% by weight of the composition.

In certain embodiments, the composition of the subject invention comprises the modified sophorolipid at, for example, about 0.001 to about 70%, about 0.01 to about 60%, about 0.1 to about 50%, about 0.1 to about 45%, about 0.1 to about 40%, about 0.1 to about 35%, about 0.1 to about 30%, about 0.1 to about 25%, about 0.1 to about 20%, about 0.1 to about 20%, about 0.1 to about 15%, about 0.1 to about 10%, about 0.1 to about 9.0%, about 0.1 to about 8.0%, about 0.1 to about 7.0%, about 0.1 to about 6.0%, about 0.1 to about 5.0%, about 0.1 to about 4.0%, about 0.1 to about 3.0%, about 0.1 to about 2.0%, about 1.0 to about 9.0%, about 1.0 to about 5.0%, about 1.0 to about 3.0%, about 3.0 to about 10%, about 3.0 to about 7.0%, about 5.0 to about 10%, about 5.0 to about 9.0%, about 6.0 to about 10%, about 7.0 to about 10%, about 8.0 to about 10%, about 5 to about 40%, about 10 to about 40%, about 10 to about 30%, about 10 to about 20%, about 20 to about 30%, about 20 to about 40%, about 20 to about 50%, about 30 to about 50%, or about 30 to about 40%.

Other active defoaming compounds, such as, silica, hydrophobed silica, polyethylene wax, ethylene bisstear amide wax, polypropylene wax, polydimethylsiloxane, organically modified polydimethylsiloxane, and hydrophobed polyethylene oxide glycerol ethers may be used in combination with the biobased antifoaming agent or foam control agent according to the invention.

Optionally, the composition can further comprise one or more other components, including, for example, carriers (e.g., water), other biosurfactants, other surfactants (e.g., polyalkyglucosides such as capryl glucoside and lauryl glucoside), hydrophilic and/or hydrophobic syndetics, sequestrants, builders (e.g., potassium carbonate, sodium hydroxide, glycerin, citric acid, lactic acid), solvents (e.g., water, ethanol, methanol, isopropanol), organic and/or inorganic acids (e.g., lactic acid, citric acid, boric acid), essential oils, botanical extracts, cross-linking agents, chelators (e.g., potassium citrate), fatty acids, alcohols, pH adjusting agents, reducing agents, calcium salts, carbonate salts, buffers, enzymes, dyes, colorants, fragrances, preservatives (e.g., octylisothiazolinone, methylisothiazolinone), terpenes (e.g., d-limonene), sesquiterpenoids, terpenoids, emulsifiers, demulsifiers, bleaching agents, polymers, thickeners and/or viscosifiers (e.g., xanthan gum, guar gum).

In some embodiments, the composition comprises additional biosurfactants. Additional biosurfactants according to the subject invention can include, for example, glycolipids, lipopeptides, flavolipids, phospholipids, fatty acid esters, and high-molecular-weight biopolymers such as lipoproteins, lipopolysaccharide-protein complexes, and/or polysaccharide-protein-fatty acid complexes.

In one embodiment, the additional biosurfactant is a glycolipid, such as, for example, rhamnolipids (RLP), cellobiose lipids, trehalose lipids and/or mannosylerythritol lipids (MEL). In one embodiment, the biosurfactant is a lipopeptide, such as, for example, surfactin, iturin, fengycin, arthrofactin, amphisin, viscosin, lichenysin, paenibacterin, polymyxin and/or battacin.

In certain embodiments, the composition further comprises a detergent auxiliary component. Preferably, the detergent auxiliary component is selected from the group consisting of an enzyme, an oxygen bleaching agent, a bleaching activator, a fluid reforming agent, and a neutral inorganic salt. Examples of the enzyme include amylase, protease, cellulose, lipase, pullulanase, isopullulanase, isoamylase, catalase, peroxidase, or the like. Examples of the oxygen bleaching agent include peroxides that generate hydrogen peroxide in an aqueous solution, such as perborate, percarbonate, persulfate and the like. The bleaching activator is used for improving a bleaching effect, such as tetra acetyl ethylene diamine (TAED), tetraacetylglycoluril (TAGU), diacetyl dioxohexahydrotriadine (DADHT), glucose penta acetate (GPA), sodium nonanoyloxybenzenesulfonate (SNOBS) or the like. The neutral inorganic salts include sodium sulfate, potassium Sulfate, and the like. The fluid reforming agent may be silica powder, anhydrous silicate or the like.

The composition may further include additional active or inactive ingredients, suitable for the mode of administration and intended purpose, provided that such addition does not adversely interfere with the functions of the modified sophorolipids.

The cleaning compositions of the subject invention can be formulated as, for example, microemulsions, dissolvable powders and/or granules, pressed powders, loose powders, diluted sprays, concentrates, aerosols, foams, toilet bowl cleaners, laundry detergents, dishwashing detergents, encapsulated dissolvable pods, gels, and/or as a pre-moistened or water-activated cloth, sponge, wipe or other substrate.

In certain embodiments, the composition may further comprise an acceptable carrier, depending on the form of the composition. The acceptable carriers include, but are not limited to, inert diluents, disintegrating agents, binding agents, lubricating agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid, or talc.

In some embodiments, the composition is in solid form and suitable for reconstitution in a solvent. In other embodiments, the composition further comprises a solvent. Suitable solvents may be aqueous or non-aqueous. In particular embodiments, the solvent is an aqueous solvent. Examples of suitable solvents include water, buffer, acetonitrile, water and alcohol mixtures such as aqueous methanol, aqueous ethanol and aqueous isopropanol.

Non-biological surfactants can also be added to the composition. Examples of surfactants include, but are not limited to, alkyl sulfates, alkyl ether sulfates (e.g., sodium/ammonium lauryl sulfates and sodium/ammonium laureth sulfates), amphoterics (e.g., amphoacetates and amphopropionates), sulfosuccinates, alkyl polyglucosides, betaines (e.g., cocamidopropyl betaine), sultaines, sacrosinates, isethionates, taurates, ethoxylated sorbitan esters, alkanolamides and amino acid-based surfactants.

Viscosity modifiers can also be added to the compositions, including, for example, cocamide DEA, oleamide DEA, sodium chloride, cellulosic polymers, polyacrylates, ethoxylated esters, alcohol, glycols, xylene sulfonates, polysorbate 20, alkanolamides, and cellulose derivatives (e.g., hydroxypropyl methylcellulose and hydroxyethyl cellulose).

Polymers can also be added, including, for example, xanthan gum, guar gum, polyquaternium-10, PEG-120 methyl glucose dioleate, PEG-150 distearate, PEG-150 polyglyceryl-2 tristearate and PEG-150 pentaerythrityl tetrastearate.

In some embodiments, the composition may optionally include a preservative. Generally, preservatives fall into specific classes including phenolics, halogen compounds, quaternary ammonium compounds, metal derivatives, amines, alkanolamines, nitro derivatives, biguanides, analides, organosulfur and sulfur-nitrogen compounds, alkyl parabens, and miscellaneous compounds. Some non-limiting examples of phenolic antimicrobial agents include pentachlorophenol, orthophenylphenol, chloroxylenol, p-chloro-m-cresol, p-chlorophenol, chlorothymol, m-cresol, o-cresol, p-cresol, isopropyl cresols, mixed cresols, phenoxyethanol, phenoxyethylparaben, phenoxyisopropanol, phenyl paraben, resorcinol, and derivatives thereof. Some non-limiting examples of halogen compounds include trichlorohydroxy diphenyl ether (Triclosan), sodium trichloroisocyanurate, sodium dichloroisocyanurate, iodine-poly(vinylpyrolidin-onen) complexes, and bromine compounds such as 2-bromo-2-nitropropane-1,3-diol, and derivatives thereof. Some non-limiting examples of quaternary ammonium compounds include benzalkonium chloride, benzethonium chloride, behentrimonium chloride, cetrimonium chloride, and derivatives thereof. Some non-limiting examples of amines and nitro containing compounds include hexahydro-1,3,5-tris(2-hydroxyethyl)-s-triazine, dithiocarbamates such as sodium dimethyldithiocarbamate, and derivatives thereof. Some non-limiting examples of biguanides include polyaminopropyl biguanide and chlorhexidine gluconate. Some non-limiting examples of alkyl parabens include methyl, ethyl, propyl and butyl parabens. The preservative is preferably present in the composition in an amount from about 0 to about 3 wt. %, from about 0.1 to about 2 wt. %, and from about 0.2 to about 1 wt. %.

The composition can include pH adjusters (e.g., citric acid, ethanolamine, sodium hydroxide, etc.) to be formulated within a wide range of pH levels. In certain embodiments, the pH of the composition ranges from 2.0 to 11.0, 2.5 to 10, 3.0 to 9.0, 3.0 to 8.0, 3.0 to 7.0, 4.0 to 7.0, 5.0 to 7.0, 6.0 to 7.0, 6.0 to 8.0, or 6.0 to 9.0. Other pH ranges suitable for the subject composition include from 3.5 to 7.0, or from 7.0 to 10.5. Suitable pH adjusters such as sodium hydroxide, citric acid and triethanolamine may be added to bring the pH within the desired range.

The composition can be placed in containers of appropriate size, taking into consideration, for example, the intended use, and the contemplated method of application. Thus, the containers into which the composition is placed may be, for example, from 0.1 gallon to 1,000 gallons or more. The composition may further be placed into smaller containers, such as bottles (e.g., 1.5 oz, 500 ml and 1 liter bottles), for distribution of individual doses of the composition.

In some embodiments, the modified sophorolipids of the present invention are provided in the form of a pharmaceutically acceptable salt. For example, the side chains of amino acids may also form a corresponding salt if it contains an appropriate functional group. Salts may be formed by procedures well known and described in the art. Examples of pharmaceutically acceptable cations for salts include, without limitation, aluminium, arginine, benzathine, calcium, chloroprocaine, choline, diethanolamine, ethanolamine, ethylenediamine, lysine, magnesium, histidine, lithium, meglumine, potassium, procaine, sodium, triethanolamine, and zinc.

Depending on the chemical characteristics of the amino acids in the modified sophorolipids, acid addition salts may be formed. Examples of pharmaceutically acceptable acid addition salts include, but are not limited to, the non-toxic inorganic and organic acid addition salts such as the acetate derived from acetic acid, the aconate derived from aconitic acid, the ascorbate derived from ascorbic acid, the benzenesulfonate derived from benzenesulfonic acid, the benzoate derived from benzoic acid, the cinnamate derived from cinnamic acid, the citrate derived from citric acid, the embonate derived from embonic acid, the enantate derived from enanthic acid, the formate derived from formic acid, the fumarate derived from fumaric acid, the glutamate derived from glutamic acid, the glycolate derived from glycolic acid, the hydrochloride derived from hydrochloric acid, the hydrobromide derived from hydrobromic acid, the lactate derived from lactic acid, the maleate derived from maleic acid, the malonate derived from malonic acid, the mandelate derived from mandelic acid, the methanesulfonate derived from methane sulphonic acid, the naphthalene-2-sulphonate derived from naphtalene-2-sulphonic acid, the nitrate derived from nitric acid, the perchlorate derived from perchloric acid, the phosphate derived from phosphoric acid, the phthalate derived from phthalic acid, the salicylate derived from salicylic acid, the sorbate derived from sorbic acid, the stearate derived from stearic acid, the succinate derived from succinic acid, the sulphate derived from sulphuric acid, the tartrate derived from tartaric acid, the toluene-p-sulphonate derived from p-toluene sulphonic acid, and the like.

The modified sophorolipids of the invention may be provided in unsolvated or solvated forms together with a pharmaceutically acceptable solvent(s) such as water, ethanol, and the like. Solvated forms may also include hydrated forms such as the monohydrate, the dihydrate, the hemihydrate, the trihydrate, the tetrahydrate, and the like.

The modified sophorolipids of the present invention, as anti-foaming agents or foam control agents, have excellent defoaming and foam inhibiting performance. The anti-foaming agent or foam control agent comprising the modified sophorolipid of the present invention may be used to inhibit or reduce the foam formation in, for example, detergents, foods, beverages, pharmaceuticals, cleaning products, industrial processes and biotechnology.

In one embodiment, the anti-foaming agent or foam control agent comprising the modified sophorolipid of the present invention may be useful in the control of foaming in painting/coating products. In on embodiment, the anti-foaming agent or foam control agent comprising the modified sorphorolipid of the present invention may be used as an additive in applications where foaming is not desired, and/or inhibition of foam formation is needed.

In one embodiment, the anti-foaming agent or foam control agent comprising the modified sophorolipid of the present invention may be used as an ingredient in foods and in materials for food preparation.

In one embodiment, the anti-foaming agent or foam control agent comprising the modified sophorolipid of the present invention may be added in the process of the industrial wastewater treatment to inhibit or reduce the foam formation.

In one embodiment, the anti-foaming agent or foam control agent comprising the modified sophorolipid of the present invention may be used in the oil and gas industry, such as in oil drilling and well treatment to inhibit or reduce the foam formation.

In general, a defoaming composition operates to lower interfacial tension in a liquid. By lowering the interfacial tension, the defoaming composition may enable gas to escape from the liquid. Accordingly, defoaming compositions of the present invention may be useful in well treatment fluids (e.g., drilling fluids, fracturing fluids, cement compositions and the like) to hinder foaming or air entrainment during agitating, mixing, or pumping such fluids.

The anti-foaming agent or foam control agent comprising the modified sophorolipid of the present invention can also be used in many other industrial processes and products such as wood pulp, paper, paint, machine tool industry, oils cutting tools, and hydraulics.

In one embodiment, the subject invention provides a method for defoaming a liquid, the method comprising combining/mixing the liquid with a defoaming composition of the subject invention, wherein the defoaming composition comprises an anti-foaming agent or foam control agent comprising the modified sophorolipid of the present invention.

In one embodiment, the subject invention provides a method for defoaming a liquid, the method comprising adding a defoaming composition of the subject invention to the liquid in need thereof, wherein the defoaming composition comprises an anti-foaming agent or foam control agent comprising the modified sophorolipid of the present invention.

In a specific embodiment, defoaming a liquid may include hindering the formation of foam or entrainment of gas in the liquid during preparation or pumping of the liquid.

In a specific embodiment, the liquid may be selected from, for example, paints, fermentation broths, detergent compositions, drilling fluids, crop protection and crop nutrient formulations, fiber finish and textile treatment formulations, fracturing fluids, fluids used in pulp/paper processing, and cement compositions.

In one embodiment, the subject invention also provides a method for preventing or reducing foam formation in a liquid, wherein the method comprises combining the liquid with the defoaming composition of the subject invention. Preferably, the liquid is selected from paints, fermentation broths, detergent compositions, drilling fluids, crop protection and crop nutrient formulations, fiber finish and textile treatment formulations, fracturing fluids, and cement compositions.

### EXAMPLE

Following are exemplary compounds of the present invention, which are offered by way of illustration and are not intended to limit the invention.

Through experimental techniques, Ferma SL and Ferma SH demonstrate lower HLB values than their calculated/theoretical HLB. This could be due to a variety of reasons including the carboxylic acid moiety of SH not playing as big of a role in HLB contributions to hydroxyl groups on sophorose being covered up by the fatty acid chain/lactone. The hydrophilic half containing sophorose, can be peracetylated to significantly reduce its hydrophilicity allowing a drop in HLB from 10 (for linear species) and 7 (for lactonic species) to below 3 that denotes an anti-foaming agent.

TABLE 1
Exemplary compounds

| Bio- | | | |
|---|---|---|---|
| Car- | | | |
| bon | | Chem- | |
| Con- | | Axon | Davies |
| tent | Compound | HLB | HLB |
| | Ferma SL (experimental HLB = 7) | | |
| 100% bio- car- bons | 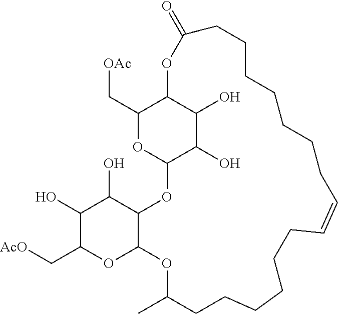 [SMILES: CC(=O)OCC1OC2OC(C)CCCCCC/C=C\CCCCCCCC(=O)OC3C(COC(C)=O)OC(OC2C(O)C1O)C(O)C3O] | 8.44 | 5.08 |
| | Ferma SH (experimental HLB = 10) | | |
| 100% bio- car- bons | 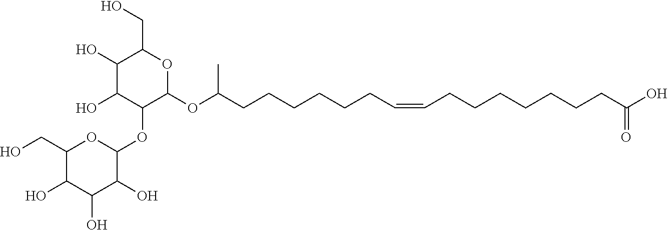 [SMILES: CC(CCCCCC/C=C\CCCCCCCC(=O)O)OC1OC(CO)C(O)C(O)C1OC1OC(CO)C(O)C(O)C1O] | 13.77 | 13.83 |
| | SH-global ether-COOH | | |
| 68.2% bio- car- bons | 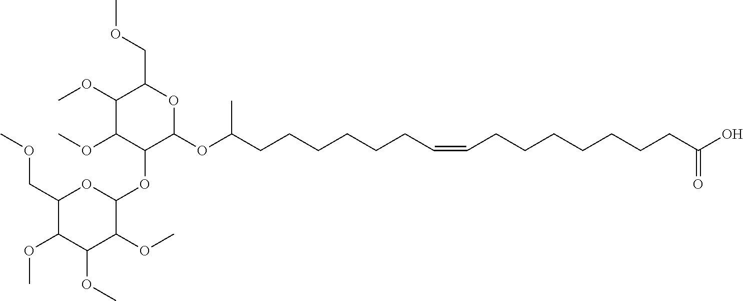 [SMILES: COCC1OC(OC2C(OC(C)CCCCCC/C=C\CCCCCCCC(=O)O)OC(COC)C(OC)C2OC)C(OC)C(OC)C1OC] | 6.92 | 2.97 |
| | SH-peracetylated-COOH | | |
| 68.2% bio- car- bons | 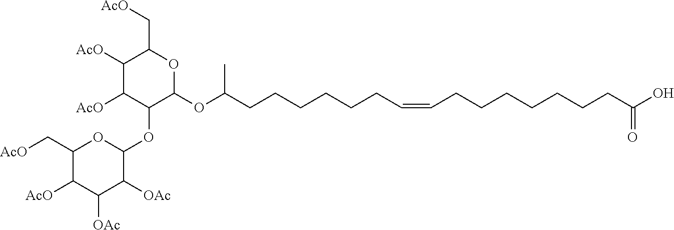 [SMILES: CC(=O)OCC1OC(OC2C(OC(C)CCCCCC/C=C\CCCCCCCC(=O)O)OC(COC(C)=O)C(OC(C)=O)C2OC(C)=O)C(OC(C)=O)C(OC(C)=O)C1OC(C)=O] | 6.07 | 1 |
| | SH-peracetylated-furfuryl | | |
| 61.3% bio- car- bons | 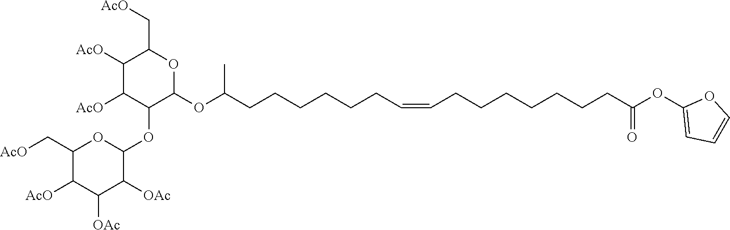 [SMILES: CC(=O)OCC1OC(OC2C(OC(C)CCCCCC/C=C\CCCCCCCC(=O)Oc3ccco3)OC(COC(C)=O)C(OC(C)=O)C2OC(C)=O)C(OC(C)=O)C(OC(C)=O)C1OC(C)=O] | 5.94 | 1 |
| | SH-global ether-furfuryl | | |
| 61.3% bio- car- bons | 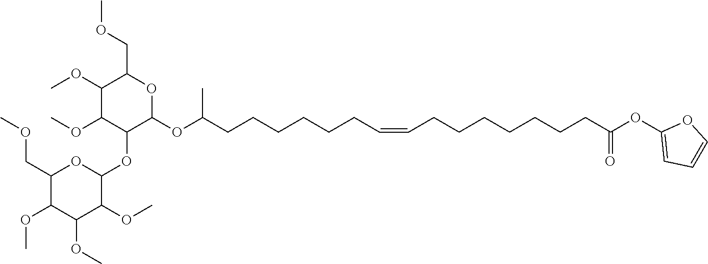 [SMILES: COCC1OC(OC2C(OC(C)CCCCCC/C=C\CCCCCCCC(=O)Oc3ccco3)OC(COC)C(OC)C2OC)C(OC)C(OC)C1OC] | 5.6 | 1 |
| | Global Ether lactonic | | |
| 71.2% bio- car- bons | 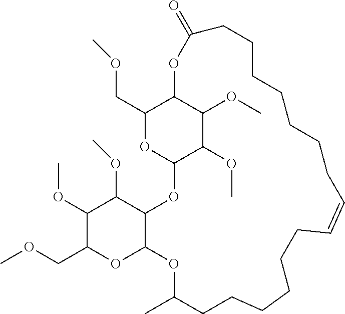 [SMILES: COCC1OC2OC3C(OC(C)CCCCCC/C=C\CCCCCCCC(=O)OC1C(OC)C2OC)OC(COC)C(OC)C3OC] | 5.85 | 1.35 |
| | SH-peracetylated-OMe | | |
| 64.4% bio- car- bons | 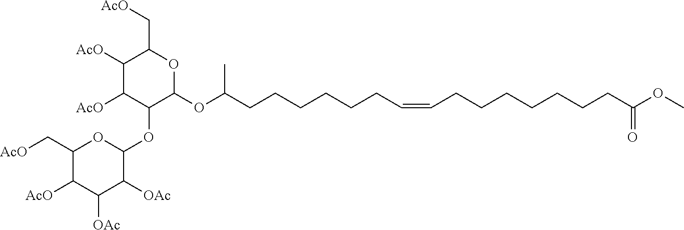 [SMILES: COC(=O)CCCCCCC/C=C\CCCCCCC(C)OC1OC(COC(C)=O)C(OC(C)=O)C(OC(C)=O)C1OC1OC(COC(C)=O)C(OC(C)=O)C(OC(C)=O)C1OC(C)=O] | 6.12 | 1 |
| | SH-peracetylated-Oet | | |
| 63.1% bio- car- bons | 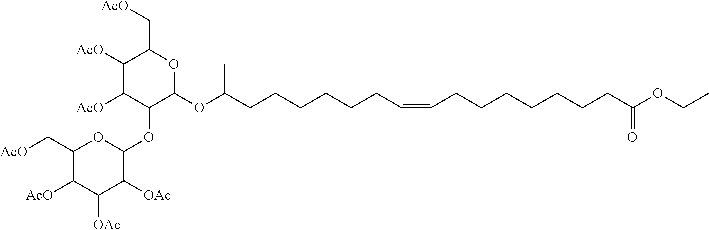 [SMILES: CCOC(=O)CCCCCCC/C=C\CCCCCCC(C)OC1OC(COC(C)=O)C(OC(C)=O)C(OC(C)=O)C1OC1OC(COC(C)=O)C(OC(C)=O)C(OC(C)=O)C1OC(C)=O] | 6.03 | 1 |
| | SH-peracetylated-O-i-bu (isobutyl) | | |
| 62.5% bio- car- bons | 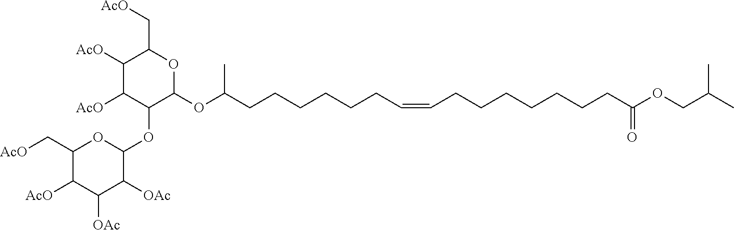 [SMILES: CC(=O)OCC1OC(OC2C(OC(C)CCCCCC/C=C\CCCCCCCC(=O)OCC(C)C)OC(COC(C)=O)C(OC(C)=O)C2OC(C)=O)C(OC(C)=O)C(OC(C)=O)C1OC(C)=O] | 5.89 | 1 |
| | SH-peracetylated-phenylalanine | | |
| 72.3% bio- car- bons | 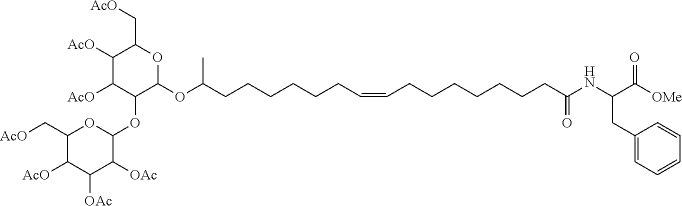 [SMILES: COC(=O)C(Cc1ccccc1)NC(=O)CCCCCCC/C=C\CCCCCCC(C)OC1OC(COC(C)=O)C(OC(C)=O)C(OC(C)=O)C1OC1OC(COC(C)=O)C(OC(C)=O)C(OC(C)=O)C1OC(C)=O] | 7.5 | 3.8 |
| | SH-peracetylated-tryptophan | | |
| 73.3% bio- car- bons | 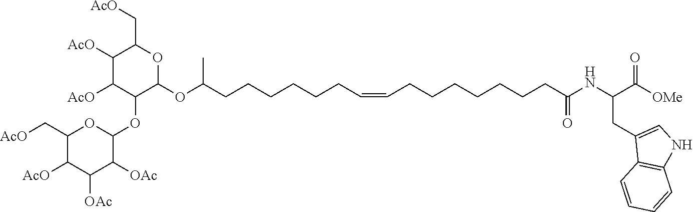 [SMILES: COC(=O)C(Cc1c[nH]c2ccccc12)NC(=O)CCCCCCC/C=C\CCCCCCC(C)OC1OC(COC(C)=O)C(OC(C)=O)C(OC(C)=O)C1OC1OC(COC(C)=O)C(OC(C)=O)C(OC(C)=O)C1OC(C)=O] | 7.28 | 3.24 |

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application

1. A compound having a structure selected from: 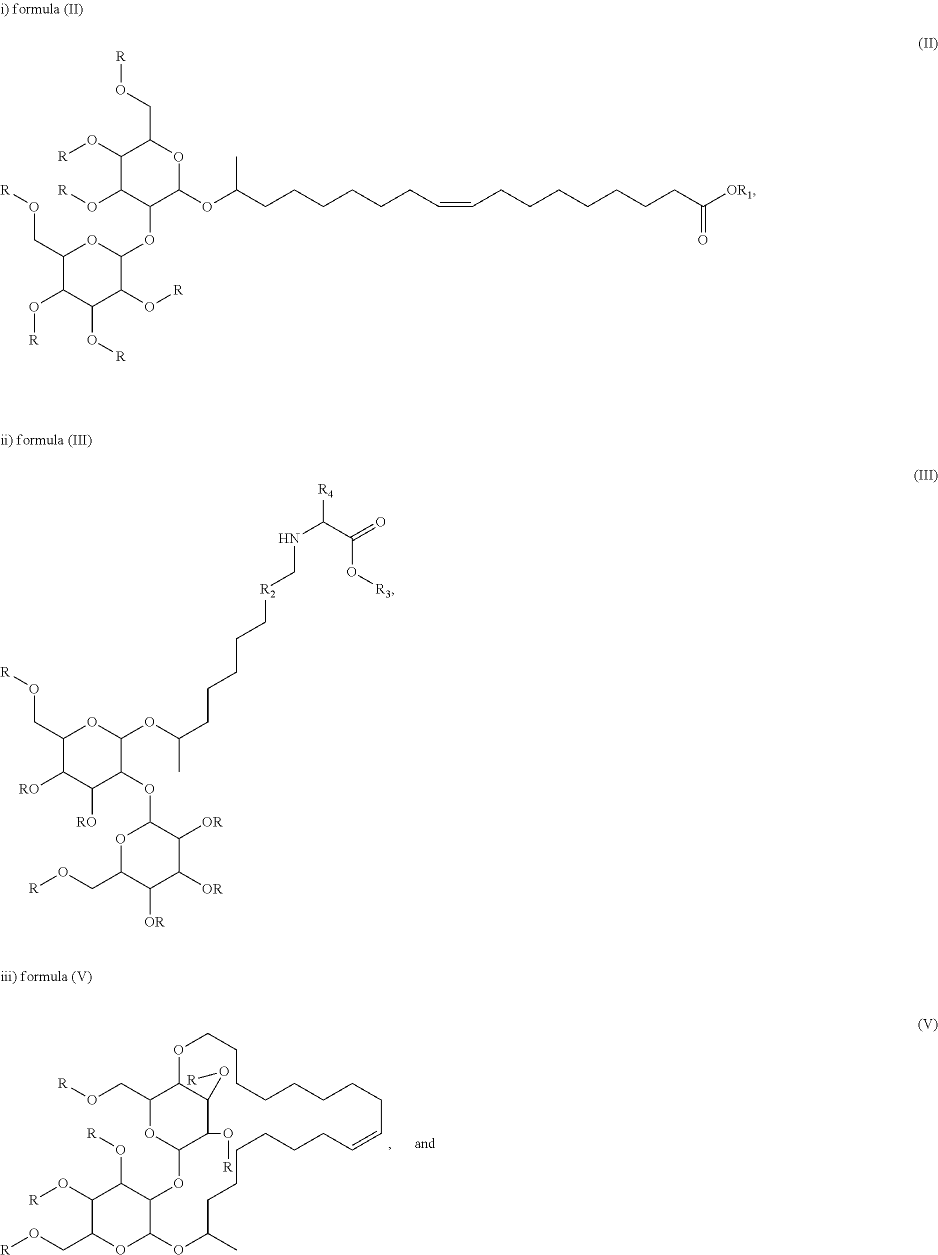 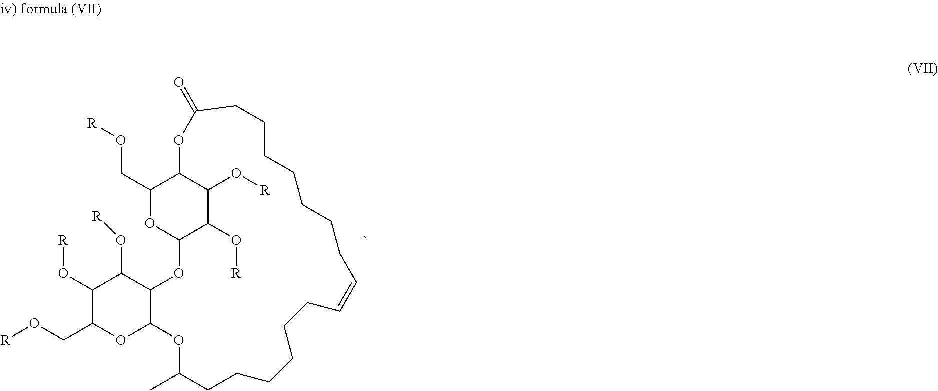
wherein each R is independently selected from hydrogen, alkyl, substituted alkyl, acyl, substituted acyl, 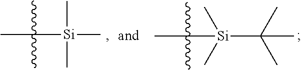
R₁ is selected from hydrogen, alkyl, substituted alkyl, heteroaryl, and substituted heteroaryl; R₂ is —(CH2)ₙ-, wherein n≥1; R₃ is selected from hydrogen, alkyl, substituted alkyl, heteroaryl, and substituted heteroaryl; and R₄ is selected from amino acid side chains.

2. The compound of claim 1, wherein each R is independently selected from H,

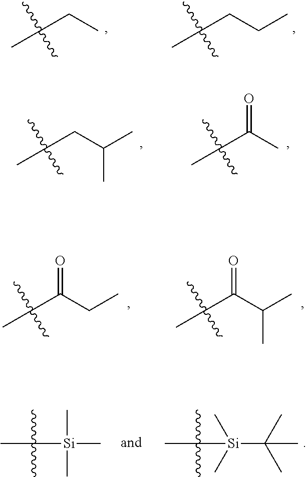

3. The compound of claim 1, wherein R₁ is selected from hydrogen, methyl, ethyl, propyl, butyl, pentyl, i-propyl, i-butyl, and

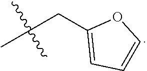 [SMILES: *Cc1ccco1]

4. The compound of claim 1, wherein R₂ is —(CH2)ₙ-, wherein 1≤n≤10.

5. The compound of claim 1, wherein R₃ is selected from hydrogen, methyl, ethyl, propyl, butyl, pentyl, i-propyl, and i-butyl.

6. The compound of claim 1, wherein R₄ is selected from

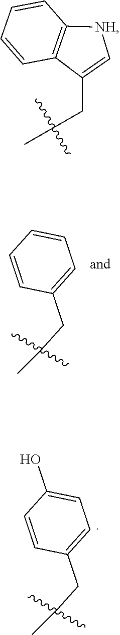 [SMILES: *Cc1c[nH]c2ccccc12 ; *Cc1ccccc1 ; *Cc1ccc(O)cc1]

7. The compound of claim 1, wherein each R is independently selected from

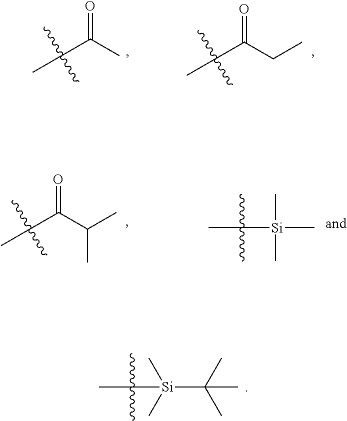

8. The compound of claim 1, which is selected from SH-global ether-COOH, SH-peracetylated-COOH, SH-peracetylated-furfuryl, SH-global ether-furfuryl, Global Ether lactonic, SH-peracetylated-OMe, SH-peracetylated-Oet, SH-peracetylated-O-i-Bu (isobutyl), SH-peracetylated-phenylalanine, and SH-peracetylated-tryptophan.

9. The compound of claim 1, which has a hydrophile-lipophile balance (HLB) value ≤4.

10. The compound of claim 1, which have a biobased carbon content of from about 50% to about 80%.

11. The compound of claim 1, which have a biobased carbon content of from about 60% to about 75%.

12. A defoaming composition comprising the compound of claim 1.

13. The defoaming composition of claim 12, further comprising a compound selected from silica, hydrophobed silica, polyethylene wax, ethylene bisstear amide wax, polypropylene wax, polydimethylsiloxane, organically modified polydimethylsiloxane, and hydrophobed polyethylene oxide glycerol ethers.

14. An anti-foaming detergent composition comprising the compound of claim 1 and a detergent auxiliary component.

15. The anti-foaming detergent composition of claim 14, wherein the detergent auxiliary component is selected from an enzyme, an oxygen bleaching agent, a bleaching activator, a fluid reforming agent, and a neutral inorganic salt.

16. A cleaning composition comprising the compound of claim 1 and an additional component selected from water, a solvent, an additional biosurfactant, an additional surfactant, a syndetic, a chelating agent, a builder, a preservative, a dye, an essential oil, a substrate, an enzyme, a disinfectant, a bleaching agent, a thickener and a viscosifier.

17. A method for defoaming a liquid, wherein the method comprises combining the liquid with the defoaming composition of claim 12.

18. The method of claim 17, wherein the liquid is selected from paints, fermentation broths, detergent compositions, drilling fluids, fracturing fluids, and cement compositions.

19. A method for preventing or reducing foam formation in a liquid, wherein the method comprises combining the liquid with the defoaming composition of claim 12.

20. The method of claim 19, wherein the liquid is selected from paints, fermentation broths, detergent compositions, drilling fluids, fracturing fluids, and cement compositions.
